(19) United States
(12) Patent Application Publication
(10) Pub. No.: US 2021/0030358 A1
(43) Pub. Date: Feb. 4, 2021

(54) STATE OF DISCOMFORT DETERMINATION DEVICE

(71) Applicant: Mitsubishi Electric Corporation, Tokyo (JP)

(72) Inventor: Isamu OGAWA, Tokyo (JP)

(73) Assignee: Mitsubishi Electric Corporation, Tokyo (JP)

(21) Appl. No.: 16/978,585

(22) PCT Filed: Mar. 9, 2018
(86) PCT No.: PCT/JP2018/009266

§ 371 Date: Sep. 4, 2020

(51) Int. Cl. A61B 5/00; A61B 5/024; A61B 5/16

(52) U.S. Cl. CPC A61B 5/4884; A61B 5/16; A61B 5/02405

(57) ABSTRACT

Included are: an action detection unit (16) detecting action information preset for each type of discomfort factor from behavior information corresponding to a discomfort factor of a user; a discomfort period estimating unit (20) acquiring an estimation condition (174) for a discomfort period (Δt) of the user corresponding to the action information and estimating the discomfort period (Δt) using history information corresponding to the estimation condition (174); a discomfort estimator (21) estimating a discomfort state of the user based on multiple pieces of biological information (X, Y) of the user; a discomfort estimator learning unit (22) estimating reaction time (tx, ty) to discomfort factors in the multiple pieces of biological information (X, Y) based on the discomfort period (Δt), and synchronizing input timing of the multiple pieces of biological information (X, Y) to the discomfort estimator (21) based on the discomfort period (Δt) and the reaction time (tx, ty); and a discomfort determination unit (19) determining the discomfort state of the user based on an estimation result of the discomfort estimator (21) when the action information is detected.

### TECHNICAL FIELD

The present invention relates to a state of discomfort determination device for determining a state of discomfort of a user on the basis of biological information of the user.

### BACKGROUND ART

In a related art, technology for determining a user's emotion on the basis of biological information is provided. A device employing such technology for determining emotions is disclosed in Patent Literature 1, for example. This Patent Literature 1 discloses a state of discomfort determination device for determining a stress state of a user on the basis of brain potential data and pulse data.

### CITATION LIST

### Patent Literature

Patent Literature 1: JP 2017-119109 A

### SUMMARY OF INVENTION

### Technical Problem

The above-mentioned state of discomfort determination device of the related art determines the stress state of a user using brain potential data and pulse data, and thus it is necessary to simultaneously acquire these two types of data. At this point, time required for acquisition of pulse data is longer than time required for acquisition of brain potential data. Therefore, the above-mentioned state of discomfort determination device of the related art solves this disadvantage by delaying the acquisition timing of the brain potential data.

However, although in the above-mentioned state of discomfort determination device of the related art, delay time for acquisition of the biological information is considered as described above, no consideration is given to delay time before the biological information appears as a response to stimulation to the user nor to individual differences in the response intensity.

The present invention has been made to solve the above-described disadvantages, and it is an object of the present invention to provide a state of discomfort determination device that can improve the accuracy of determining a user's state of discomfort.

### Solution to Problem

A state of discomfort determination device according to the present invention includes: an action detection unit detecting action information regarding a user's action preset for each type of discomfort factor from behavior information regarding behavior that corresponds to a discomfort factor of the user; a discomfort period estimating unit acquiring an estimation condition for a discomfort period of the user that corresponds to the action information detected by the action detection unit and estimating the discomfort period using history information that corresponds to the estimation condition; a discomfort estimator estimating a state of discomfort of the user on the basis of multiple pieces of biological information of the user; a discomfort estimator learning unit estimating reaction time to a discomfort factor in each of the multiple pieces of biological information on the basis of the discomfort period estimated by the discomfort period estimating unit, and synchronizing input timing of the multiple pieces of biological information to the discomfort estimator on a basis of the discomfort period and the reaction time; and a discomfort determination unit determining the state of discomfort of the user on the basis of an estimation result of the discomfort estimator in a case where the action detection unit detects the action information.

### Advantageous Effects of Invention

According to this invention, the accuracy of determining a user's state of discomfort can be improved.

### BRIEF DESCRIPTION OF DRAWINGS

FIG. 1 is a block diagram illustrating a configuration of a state of discomfort determination device according to a first embodiment of the invention.

FIG. 2 is a block diagram illustrating a hardware configuration of the state of discomfort determination device according to the first embodiment of the invention.

FIG. 3 is a table illustrating storage example in an action information database.

FIG. 4 is a table illustrating storage example in a control information database.

FIG. 5 is a table illustrating storage example in a learning database.

FIG. 6 is a table illustrating another storage example in the learning database.

FIG. 7 is a table illustrating storage example in an estimation parameter storing unit.

FIG. 8 is a flowchart illustrating the operation of the state of discomfort determination device according to the first embodiment of the invention.

FIG. 9 is a flowchart illustrating the operation of an environmental information acquiring unit.

FIG. 10 is a flowchart illustrating the operation of a behavior information acquiring unit.

FIG. 11 is a flowchart illustrating the operation of a biological information acquiring unit.

FIG. 12 is a flowchart illustrating the operation of a control information acquiring unit.

FIG. 13 is a flowchart illustrating the operation of an action detection unit.

FIG. 14 is a flowchart illustrating the operation of a discomfort determination unit.

FIG. 15 is a flowchart illustrating the operation of a discomfort period estimating unit.

FIG. 16 is a flowchart illustrating the operation of a discomfort estimator learning unit.

FIG. 17 is a flowchart illustrating the operation of estimating reaction time in the discomfort estimator learning unit.

FIG. 18 is a flowchart illustrating the operation of a discomfort estimator.

FIG. 19 is a time chart illustrating an example of learning of the discomfort estimator.

FIG. 20 is a time chart illustrating an example of discomfort determination by the discomfort determination unit.

### DESCRIPTION OF EMBODIMENTS

To describe the present invention further in detail, an embodiment for carrying out the present invention will be described below with reference to the accompanying drawings.

### First Embodiment

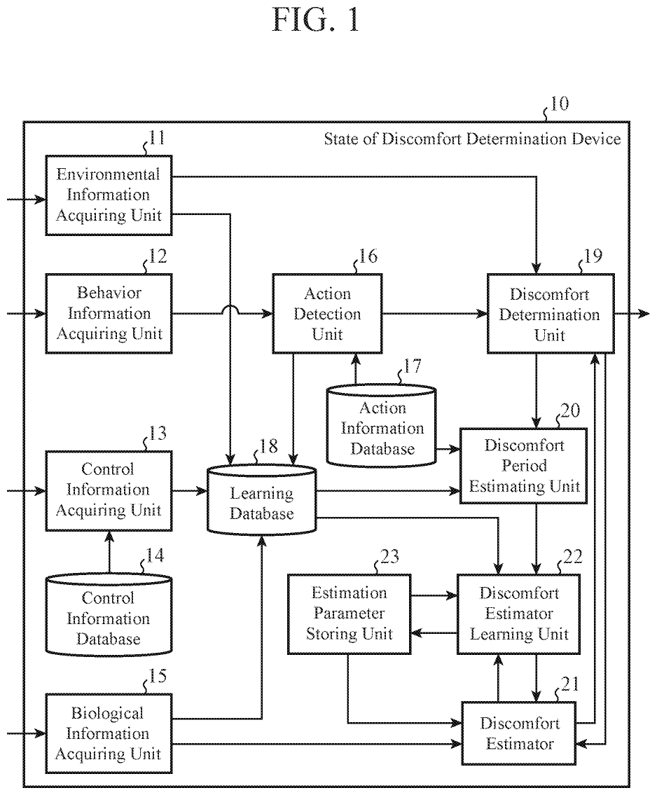

FIG. 1 is a block diagram illustrating a configuration of a state of discomfort determination device 10 according to a first embodiment of the invention.

As illustrated in FIG. 1, the state of discomfort determination device 10 includes an environmental information acquiring unit 11, a behavior information acquiring unit 12, a control information acquiring unit 13, a control information database 14, a biological information acquiring unit 15, an action detection unit 16, an action information database 17, a learning database 18, a discomfort determination unit 19, a discomfort period estimating unit 20, a discomfort estimator 21, a discomfort estimator learning unit 22, and an estimation parameter storing unit 23.

The environmental information acquiring unit 11 acquires environmental information regarding the state of the environment around a user. The environmental information includes, for example, temperature information regarding the temperature detected by a temperature sensor and noise information regarding the magnitude of noise detected by a microphone.

The behavior information acquiring unit 12 acquires behavior information regarding the action of the user. The behavior information includes, for example, image information regarding the motion of the user's face and body imaged by a camera, audio information regarding the user's voice and utterance contents detected by a microphone, and operation information regarding a user's operation of a device that is detected by an operation unit such as a touch panel and a switch.

The control information acquiring unit 13 acquires, from external devices, control information for controlling the external devices that operate on the basis of an estimation result of the state of discomfort determination device 10. The external devices include, for example, an air conditioning device and an audio device. The control information acquiring unit 13 further collates the acquired control information with control patterns stored in advance in the control information database 14 which will be described later.

The control information database 14 stores, in advance, control patterns and discomfort factors of the user that cause the control in association with each other as control information for controlling the air conditioning device and the audio device. The control patterns for controlling the air conditioning device include, for example, information regarding turning ON, turning OFF of cooling or heating, or the like. The control patterns for controlling the audio device include, for example, information regarding a volume increase or decrease. The discomfort factors that make the user feel discomfort are stimuli to the user such as hot, cold, and noisy.

The biological information acquiring unit 15 acquires multiple pieces of biological information of the user from biological sensors. The biological sensor includes, for example, a heart rate monitor and an electroencephalograph. The biological information includes, for example, information regarding heart rate variability measured by the heart rate monitor and information regarding the brain wave measured by the electroencephalograph.

The action detection unit 16 collates the behavior information acquired by the behavior information acquiring unit 12 with action patterns stored in advance in the action information database 17 described later.

The action information database 17 stores, in advance in association with one another, discomfort factors, action patterns defined in advance for each type of the discomfort factors, and estimation conditions for a discomfort period in which the user feels discomfort. Examples of action pattern include action patterns of the user such as to utter “hot” or to press a button for lowering the preset temperature of the air conditioning device in response to discomfort factors of the user of “air conditioning (hot)”. The estimation conditions for a discomfort period are, for example, the temperature in the environment and the magnitude of noise in the environment.

The learning database 18 stores the environmental information acquired by the environmental information acquiring unit 11, action patterns that match the action patterns stored in the action information database 17 through the collation operation of the action detection unit 16, the control information acquired by the control information acquiring unit 13, the biological information acquired by the biological information acquiring unit 15, time stamps, and other information.

The discomfort determination unit 19 outputs, to the outside, a signal indicating detection of a state of discomfort of the user when an action pattern that matches an action pattern stored in the action information database 17 through the matching operation of the action detection unit 16 is input thereto from the action detection unit 16. The discomfort determination unit 19 outputs the action pattern input thereto from the action detection unit 16 to the discomfort period estimating unit 20 described later. Furthermore, when a signal indicating detection of a user's state of discomfort is input from the discomfort estimator 21 described later, the discomfort determination unit 19 outputs the signal to the outside.

The discomfort period estimating unit 20 acquires estimation conditions for a discomfort period stored in the action information database 17 that correspond to the action pattern input from the discomfort determination unit 19. The discomfort period estimating unit 20 further estimates the discomfort period on the basis of the acquired estimation conditions for the discomfort period and the history information stored in the learning database 18. That is, the history information refers to the progress history of the above-described environmental information, action patterns, control information, biological information, and the time stamps.

The discomfort estimator 21 estimates whether the biological information input from the biological information acquiring unit 15 is in a state of discomfort or a normal state on the basis of the reaction time of the biological information, a normal state threshold value, and a discomfort state threshold value, which are stored in the estimation parameter storing unit 23 described later.

When the control pattern that corresponds to the discomfort factor of the discomfort period estimated by the discomfort period estimating unit 20 is input from the control information acquiring unit 13, the discomfort estimator learning unit 22 estimates, as the reaction time of the biological information, the elapsed time from the time when the control pattern is input to the time when the biological information acquired by the biological information acquiring unit 15 is changed from the state of discomfort to the normal state. Moreover, the discomfort estimator learning unit 22 performs learning of the discomfort estimator 21 on the basis of the reaction time estimated for each piece of biological information. Furthermore, the discomfort estimator learning unit 22 stores the estimated reaction time for each piece of biological information, the normal state threshold value, and the discomfort state threshold value in the estimation parameter storing unit 23 described later.

Here, the learning of the discomfort estimator 21 performed by the discomfort estimator learning unit 22 is performed to synchronize input timing to the discomfort estimator 21 of a signal indicating the heart rate variability and a signal indicating the brain wave, on the basis of the reaction time of the heart rate variability and the reaction time of the brain wave.

The estimation parameter storing unit 23 stores the reaction time of the biological information estimated by the discomfort estimator learning unit 22, the normal state threshold value, and the discomfort state threshold value for each type of the biological information of the user.

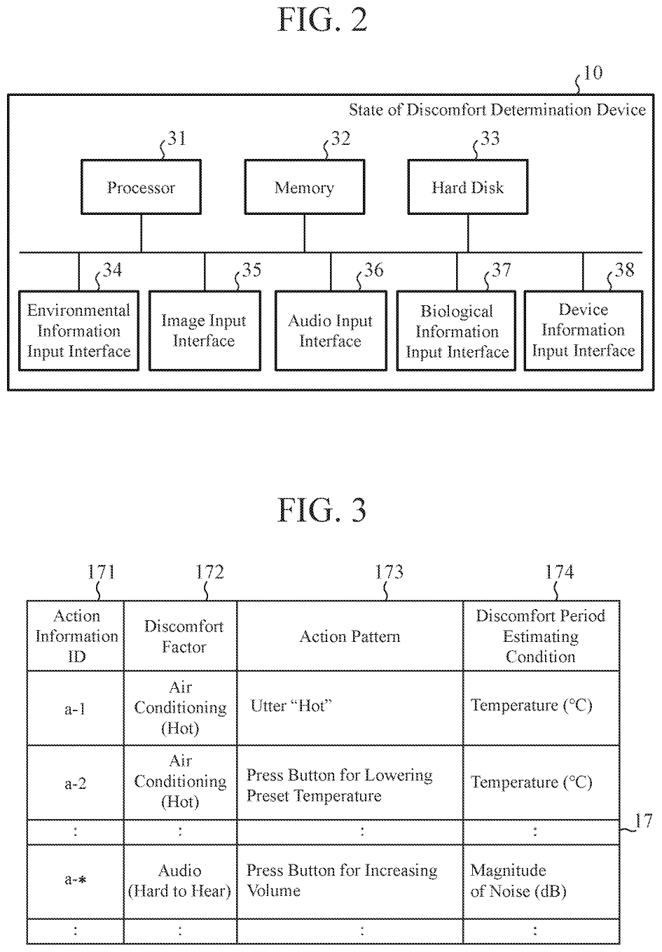

FIG. 2 is a block diagram illustrating a hardware configuration of the state of discomfort determination device 10 according to the first embodiment of the invention.

The state of discomfort determination device 10 includes a processor 31, a memory 32, a hard disk 33, an environmental information input interface 34, an image input interface 35, an audio input interface 36, a biological information input interface 37, and a device information input interface 38.

The environmental information input interface 34 includes a temperature sensor and a microphone. The image input interface 35 includes a camera. The audio input interface 36 includes a microphone. The biological information input interface 37 includes the heart rate monitor and the electroencephalograph. The device information input interface 38 includes a touch panel, a switch, and a communication device with the air conditioning device and with the audio device.

The state of discomfort determination device 10 includes a computer, and stores the control information database 14, the action information database 17, the learning database 18, and the estimation parameter storing unit 23 in the hard disk 33. In the state of discomfort determination device 10, programs are stored in the memory 32 which cause the processor 31 to function as the environmental information acquiring unit 11, the behavior information acquiring unit 12, the control information acquiring unit 13, the biological information acquiring unit 15, the action detection unit 16, the discomfort determination unit 19, the discomfort period estimating unit 20, the discomfort estimator 21, and the discomfort estimator learning unit 22. The processor 31 executes the programs stored in the memory 32.

Next, storage example in the control information database 14, the action information database 17, the learning database 18, and the estimation parameter storing unit 23 will be described in detail with reference to FIG. 3 to FIG. 7.

FIG. 3 is a table illustrating storage example in the action information database 17. As illustrated in FIG. 3, the action information database 17 stores action information IDs 171 for identifying action information, discomfort factors 172 that the user feels discomfort with, action patterns 173 that correspond to the discomfort factors, and discomfort period estimating conditions 174 in association with each other.

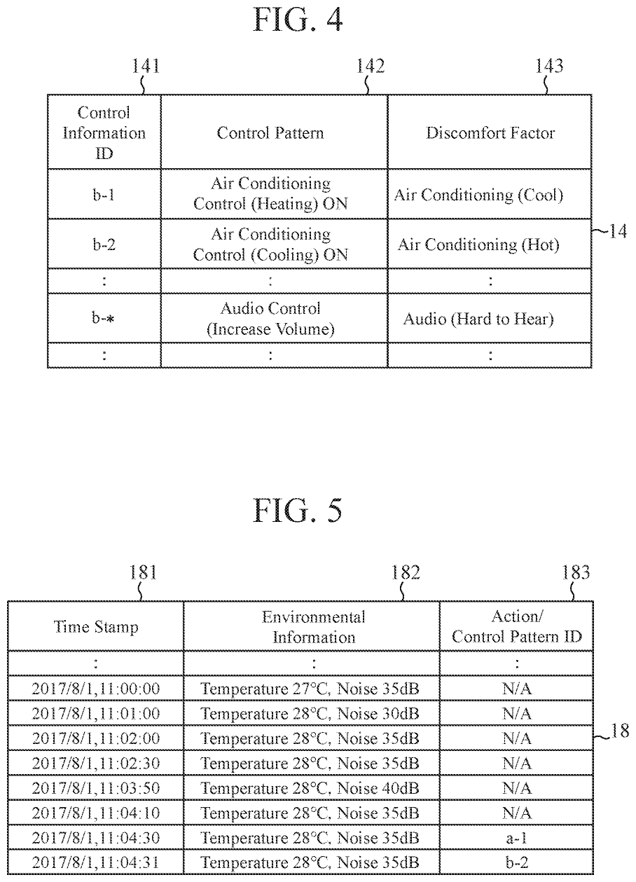

FIG. 4 is a table illustrating storage example in the control information database 14. As illustrated in FIG. 4, the control information database 14 stores control information IDs 141 for identifying control information, control patterns 142 corresponding to a user's discomfort factor in the air conditioning device or the audio device, and discomfort factors 143 that the user feels discomfort with in association with each other.

FIG. 5 and FIG. 6 are tables illustrating storage example in the learning database 18. As illustrated in FIG. 5, the learning database 18 stores time stamps 181, environmental information 182, and action/control pattern IDs 183 for identifying an action pattern or a control pattern in association with each other. In addition, as illustrated in FIG. 6, the learning database 18 stores user IDs 184 for identifying a user, types of biological information 185, acquisition start time 186 indicating time at which acquisition of a measurement value of biological information is started, and measurement values of biological information 187 in association with each other.

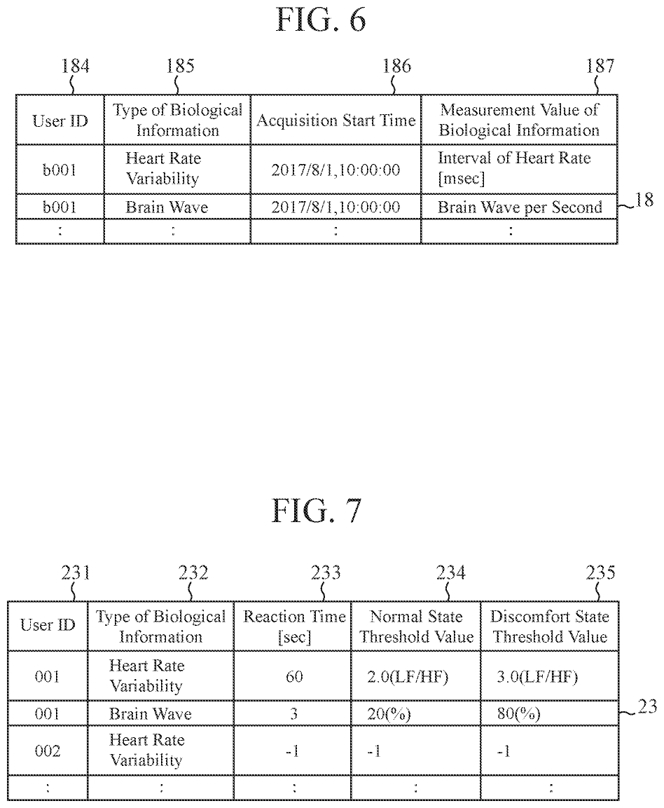

FIG. 7 is a table illustrating storage example in the estimation parameter storing unit 23. As illustrated in FIG. 7, the estimation parameter storing unit 23 stores user IDs 231 for identifying a user, types of biological information 232, reaction time of biological information 233, normal state threshold values 234, and discomfort state threshold values 235 in association with each other as estimation parameters.

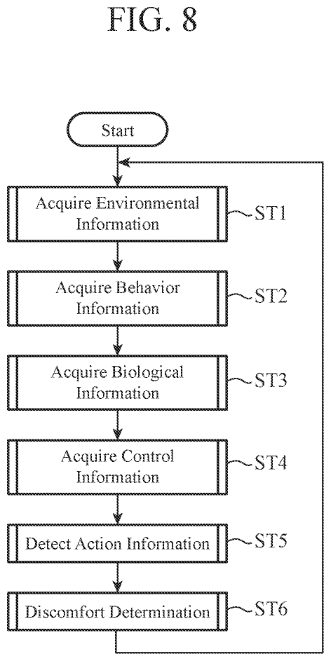

Next, the operation of the state of discomfort determination device 10 will be described in detail with reference to FIG. 8. FIG. 8 is a flowchart illustrating the operation of the state of discomfort determination device 10 according to the first embodiment. Note that the operation of the state of discomfort determination device 10 is performed in a constant cycle.

In step ST1, the environmental information acquiring unit 11 acquires, as environmental information, temperature information regarding the temperature detected by the temperature sensor and noise information regarding the magnitude of the noise detected by the microphone.

In step ST2, the behavior information acquiring unit 12 acquires, as behavior information, image information regarding the motion of the user's face and body imaged by the camera, audio information regarding the user's voice and utterance content detected by the microphone, and operation information regarding the user's operation of a device that is detected by an operation unit such as the touch panel and the switch.

In step ST3, the biological information acquiring unit 15 acquires, as biological information, information regarding heart rate variability measured by the heart rate monitor and information regarding the brain wave measured by the electroencephalograph.

In step ST4, the control information acquiring unit 13 acquires control information for controlling the air conditioning device and the audio device.

In step ST5, the action detection unit 16 detects action information from the behavior information acquired by the behavior information acquiring unit 12.

In step ST6, when the action information detected by the action detection unit 16 and the estimation result output by the discomfort estimator 21 are input, the discomfort determination unit 19 determines that the user is in a state of discomfort.

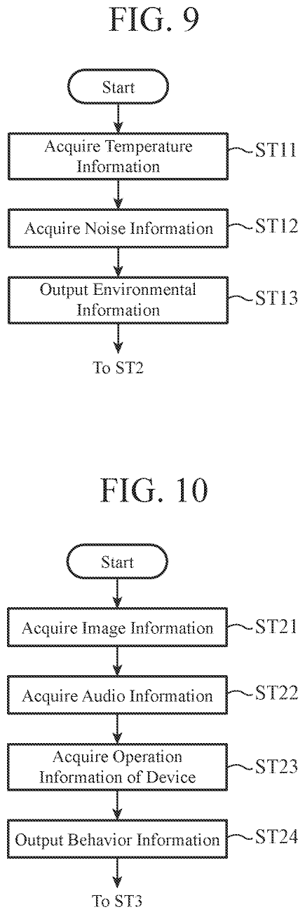

Next, the process of step ST1 will be described in more detail with reference to FIG. 9. FIG. 9 is a flowchart illustrating the operation of the environmental information acquiring unit 11.

In step ST11, the environmental information acquiring unit 11 acquires temperature information regarding the temperature detected by the temperature sensor.

In step ST12, the environmental information acquiring unit 11 acquires noise information regarding the magnitude of the noise detected by the microphone.

In step ST13, the environmental information acquiring unit 11 outputs the acquired temperature information and noise information to the learning database 18 and the discomfort determination unit 19. As a result, as illustrated in FIG. 5, the learning database 18 stores the time at which the two pieces of information are input as a time stamp 181 and stores the two pieces of input information input thereto as environmental information 182. Then, the process of the state of discomfort determination device 10 proceeds to step ST2.

Next, the process of step ST2 will be described in more detail with reference to FIG. 10. FIG. 10 is a flowchart illustrating the operation of the behavior information acquiring unit 12.

In step ST21, the behavior information acquiring unit 12 acquires image information regarding the motion of the user's face and body obtained by analyzing image signals input from the camera.

In step ST22, the behavior information acquiring unit 12 acquires audio information regarding the user's voice and utterance content obtained by analyzing the audio signal input from the microphone.

In step ST23, the behavior information acquiring unit 12 acquires operation information regarding the user's operation of a device detected by an operation unit such as the touch panel and the switch.

In step ST24, the behavior information acquiring unit 12 outputs the acquired image information, audio information, and operation information to the action detection unit 16 as the behavior information. Then, the process of the state of discomfort determination device 10 proceeds to step ST3.

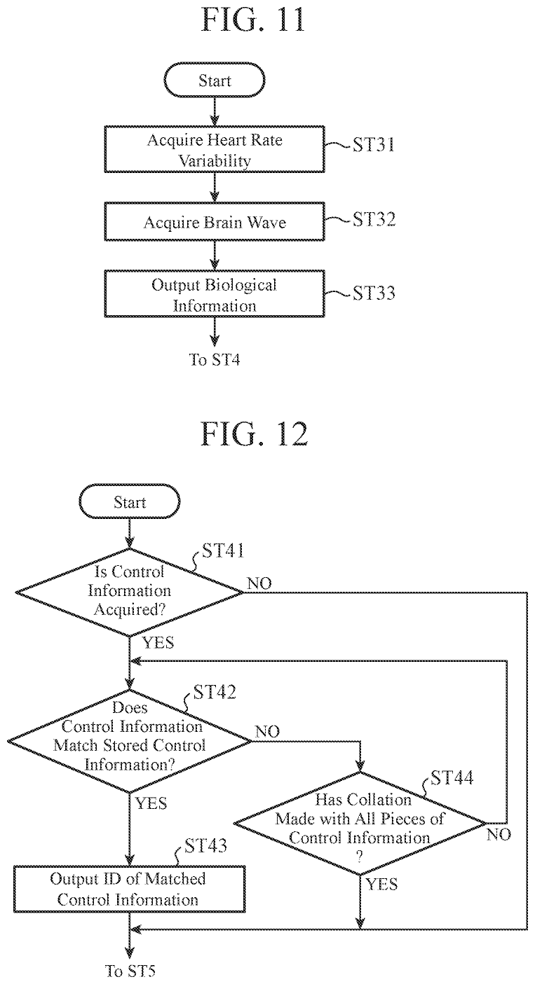

Next, the process of step ST3 will be described in more detail with reference to FIG. 11. FIG. 11 is a flowchart illustrating the operation of the biological information acquiring unit 15.

In step ST31, the biological information acquiring unit 15 acquires information regarding the heart rate variability measured by the heart rate monitor.

In step ST32, the biological information acquiring unit 15 acquires information regarding the brain wave measured by the electroencephalograph.

In step ST33, the biological information acquiring unit 15 outputs the above-described acquired two pieces of information as the biological information to the learning database 18 and the discomfort estimator 21. Then, the process of the state of discomfort determination device 10 proceeds to step ST4.

Next, the process of step ST4 will be described in more detail with reference to FIG. 12. FIG. 12 is a flowchart illustrating the operation of the control information acquiring unit 13.

In step ST41, the control information acquiring unit 13 determines whether or not the control information acquiring unit 13 acquired control information. If the control information acquiring unit 13 acquired control information, the process proceeds to step ST42. On the other hand, if the control information acquiring unit 13 has not acquired control information, the process ends. That is, the process of the state of discomfort determination device 10 proceeds to step ST5.

In step ST42, the control information acquiring unit 13 determines whether or not the acquired control information matches control information stored in the control information database 14. If the control information acquiring unit 13 determines that they match, the process proceeds to step ST43. On the other hand, if the control information acquiring unit 13 determines that they do not match, the process proceeds to step ST44.

For example, in a case where the acquired control pattern is “air conditioning control (cooling) ON”, the control information acquiring unit 13 determines that the acquired control pattern matches the control pattern having a control information ID 141 of “b-2” illustrated in FIG. 4.

In step ST43, the control information acquiring unit 13 outputs the control information ID 141 of the control information that matches the acquired control information from the control information database 14 to the learning database 18. Then, the process of the state of discomfort determination device 10 proceeds to step ST5.

Meanwhile, in step ST44, the control information acquiring unit 13 determines whether or not the acquired control information is collated with all the pieces of control information stored in the control information database 14. If the control information acquiring unit 13 determines that collation with all the pieces of control information is performed, the process ends. That is, the process of the state of discomfort determination device 10 proceeds to step ST5. On the other hand, if the control information acquiring unit 13 determines that collation with some pieces of control information is not performed, the process returns to step ST42. That is, the control information acquiring unit 13 starts collation of the acquired control information with all the remaining pieces of control information stored in the control information database 14.

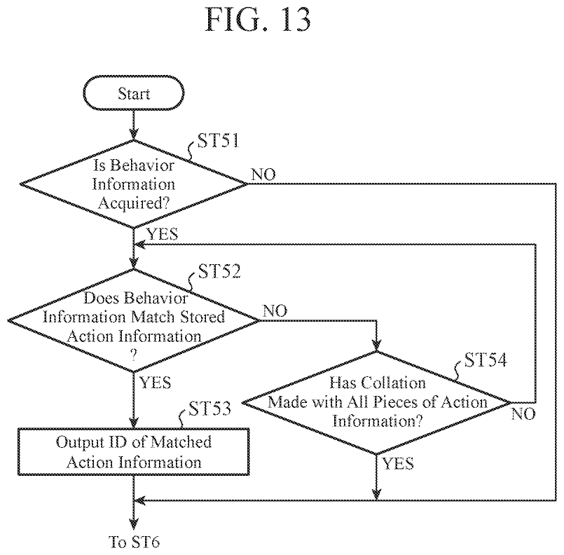

Next, the process of step ST5 will be described in more detail with reference to FIG. 13. FIG. 13 is a flowchart illustrating the operation of the action detection unit 16.

In step ST51, the action detection unit 16 determines whether or not the action detection unit 16 acquired behavior information. If the action detection unit 16 acquired behavior information, the process proceeds to step ST52. On the other hand, if the action detection unit 16 has not acquired behavior information, the process ends. That is, the process of the state of discomfort determination device 10 proceeds to step ST6.

In step ST52, the action detection unit 16 determines whether or not the acquired behavior information matches action information stored in the action information database 17. If the action detection unit 16 determines that they match, the process proceeds to step ST53. On the other hand, if the action detection unit 16 determines that they do not match, the process proceeds to step ST54.

For example, in a case where an acquired action pattern is utterance “hot” by the user, the action detection unit 16 determines that the acquired action pattern matches the action pattern 173 having an action information ID 171 of “a-1” illustrated in FIG. 3.

In step ST53, the action detection unit 16 outputs the action information ID 171 of the action information that matches the acquired behavior information from the action information database 17 to the learning database 18 and the discomfort determination unit 19. Then, the process of the state of discomfort determination device 10 proceeds to step ST6.

On the other hand, in step ST54, the action detection unit 16 determines whether or not the acquired behavior information are collated with all the pieces of action information stored in the action information database 17. If the action detection unit 16 determines that collation with all the pieces of action information are performed, the process ends. That is, the process of the state of discomfort determination device 10 proceeds to step ST6. On the other hand, if the action detection unit 16 determines that collation with some pieces of action information are not performed, the process returns to step ST52. That is, the action detection unit 16 starts collation of the acquired behavior information with all the remaining pieces of action information stored in the action information database 17.

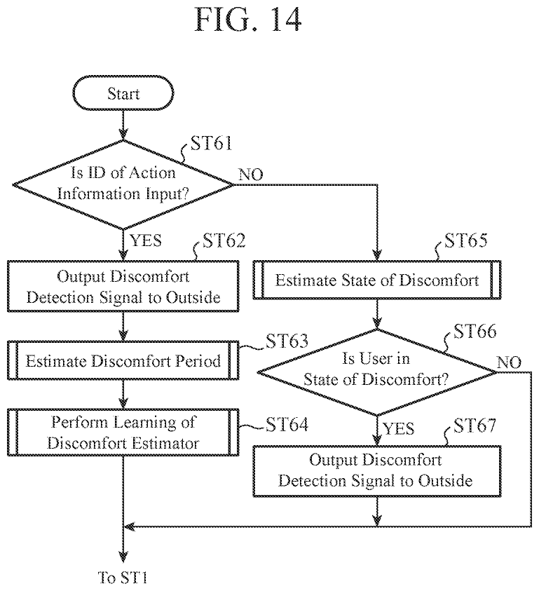

Next, the process of step ST6 will be described in more detail with reference to FIG. 14. FIG. 14 is a flowchart illustrating the operation of the discomfort determination unit 19.

In step ST61, the discomfort determination unit 19 determines whether or not an action information ID 171 stored in the action information database 17 is acquired. If the discomfort determination unit 19 acquired an action information ID 171, the process proceeds to step ST62. On the other hand, if the discomfort determination unit 19 has not acquired any action information ID 171, the process proceeds to step ST65.

In step ST62, the discomfort determination unit 19 outputs a discomfort detection signal indicating that a state of discomfort of the user is detected to the outside.

In step ST63, the discomfort determination unit 19 outputs the acquired action information ID 171 to the discomfort period estimating unit 20. Subsequently, the discomfort period estimating unit 20 estimates a discomfort period on the basis of the input action information ID 171 and outputs the estimated discomfort period to the discomfort estimator learning unit 22.

In step ST64, the discomfort estimator learning unit 22 performs leaning of the discomfort estimator 21 when the discomfort period is input from the discomfort period estimating unit 20. Then, the process of the state of discomfort determination device 10 returns to step ST1.

On the other hand, in step ST65, the discomfort estimator 21 estimates a state of discomfort of the user on the basis of the biological information input from the biological information acquiring unit 15.

In step ST66, the discomfort estimator 21 determines whether or not the user is in a state of discomfort. If the discomfort estimator 21 determines that the user is in the state of discomfort, the process proceeds to step ST67. On the other hand, if the discomfort estimator 21 determines that the user is not in the state of discomfort, the process ends. That is, the process of the state of discomfort determination device 10 returns to step ST1.

In step ST67, the discomfort determination unit 19 outputs a discomfort detection signal indicating that the state of discomfort of the user is detected to the outside. Then, the process of the state of discomfort determination device 10 returns to step ST1.

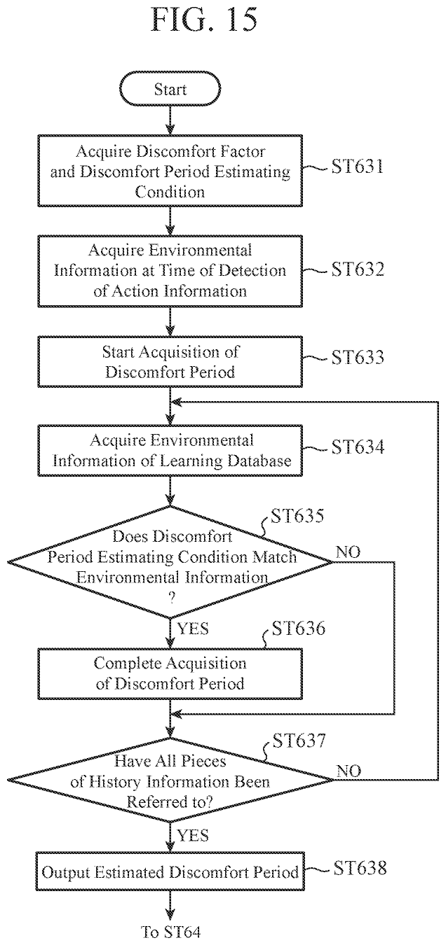

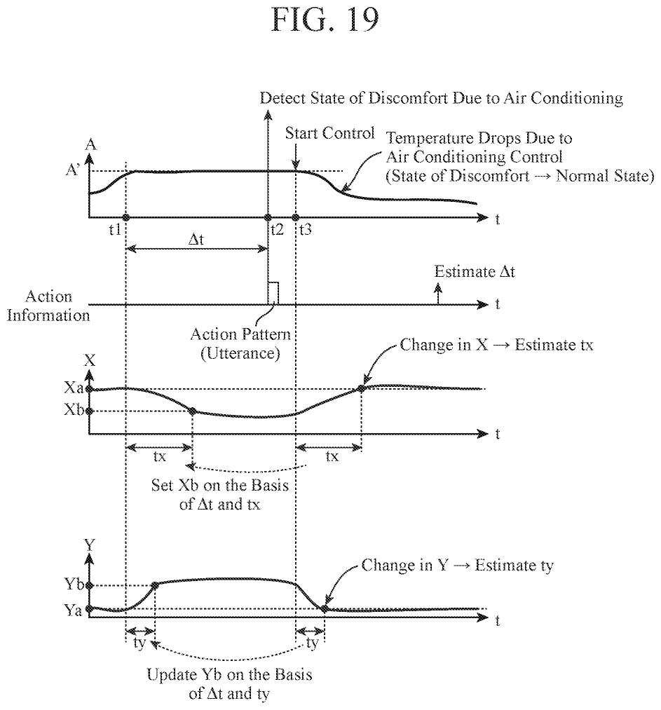

Next, the process of step ST63 will be described in more detail with reference to FIGS. 3, 5, 15, and 19. FIG. 15 is a flowchart illustrating the operation of the discomfort period estimating unit 20. FIG. 19 is a time chart illustrating an example of learning of the discomfort estimator 21. Note that “t” in FIG. 19 represents time, and “A” represents the temperature of the environment around the user.

In step ST631, the discomfort period estimating unit 20 extracts the same action information ID as the action information ID 171 input thereto from the plurality of action information IDs 171 stored in the action information database 17, and acquires the discomfort factor 172 and the discomfort period estimating condition 174 that corresponds to the extracted action information ID 171.

For example, as illustrated in FIG. 3, in a case where the discomfort period estimating unit 20 acquires “a-1” as the action information ID 171, the discomfort period estimating unit 20 searches action information having an action information ID 171 of “a-1” from among the plurality of action information IDs 171 stored in the action information database 17. Then, the discomfort period estimating unit 20 refers to the discomfort period estimating condition 174 having the action information ID 171 of “a-1”, and acquires “temperature (° C.)”.

In step ST632, the discomfort period estimating unit 20 acquires the most recent environmental information 182 stored in the learning database 18.

For example, as illustrated in FIG. 5, the discomfort period estimating unit 20 refers to the environmental information 182 stored in the learning database 18 and acquires “temperature 28° C.”.

In step ST633, the discomfort period estimating unit 20 acquires the time stamp 181 that corresponds to the most recent environmental information 182 as end time t2 of a discomfort period Δt illustrated in FIG. 19.

In step ST634, the discomfort period estimating unit 20 goes back through the history of the environmental information 182 stored in the learning database 18 and acquires the history as history information.

In step ST635, the discomfort period estimating unit 20 determines whether or not any one piece of the acquired history of the environmental information 182 matches the discomfort period estimating condition 174 acquired in step ST631. If the discomfort period estimating unit 20 determines that there is a match, the process proceeds to step ST636. On the other hand, if the discomfort period estimating unit 20 determines that there is no match, the process proceeds to step ST637.

In step ST636, the discomfort period estimating unit 20 acquires, as the discomfort period Δt illustrated in FIG. 19, a difference between time indicated by a time stamp 181 and the end time t2, which corresponds to environmental information 182 that matches the discomfort period estimating condition 174 acquired in step ST631.

In step ST637, the discomfort period estimating unit 20 determines whether or not the entire history of the environmental information 182 is referred to with respect to the acquired discomfort period estimating condition 174. If the discomfort period estimating unit 20 determines that the entire history of the environmental information 182 is referred to, the process proceeds to step ST638. On the other hand, if the discomfort period estimating unit 20 determines that not the entire history of the environmental information 182 is referred to, the process returns to step ST634.

In step ST638, the discomfort period estimating unit 20 outputs the finally acquired discomfort period Δt to the discomfort estimator learning unit 22. Then, the process of the state of discomfort determination device 10 proceeds to step ST64.

For example, as illustrated in FIG. 19, the discomfort period estimating unit 20 estimates, as a discomfort period Δt, a period from the time t2 when an action pattern 173 of the utterance “hot” by the user is detected back time t1 when the temperature becomes less than or equal to a preset temperature upper limit value A′ of 28° C., and outputs this estimated discomfort period Δt to the discomfort estimator learning unit 22.

That is, the time t1 is the start time of the discomfort period Δt, and is hereinafter referred to as start time t1. The start time t1 also serves as reference time for discomfort determination, which will be described later. The time t2 is the end time of the discomfort period Δt, and is hereinafter referred to as end time t2.

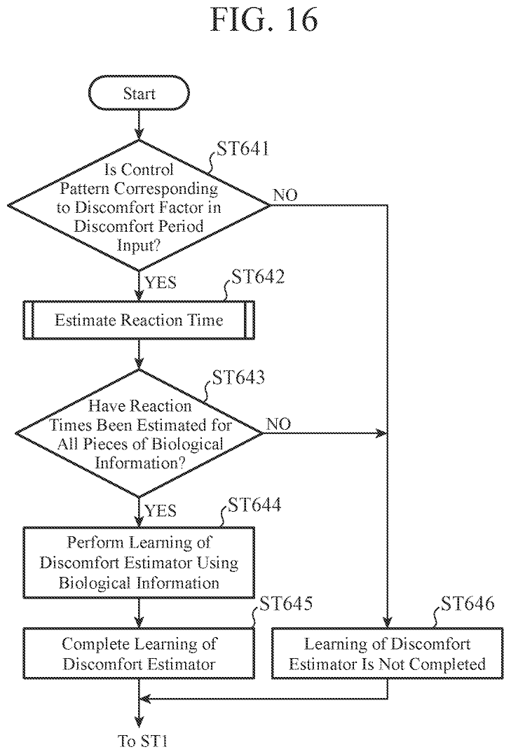

Next, the process of step ST64 will be described in more detail with reference to FIGS. 3, 4, 5, 7, 16, and 19. FIG. 16 is a flowchart illustrating the operation of the discomfort estimator learning unit 22.

In step ST641, the discomfort estimator learning unit 22 refers to the history information stored in the learning database 18 when the discomfort period Δt is input from the discomfort period estimating unit 20, and determines whether or not a control pattern 142 that corresponds to a discomfort factor 143 of the discomfort period Δt is input. If the discomfort estimator learning unit 22 determines that the control pattern 142 is input, the process proceeds to step ST642. On the other hand, if the discomfort estimator learning unit 22 determines that no control pattern 142 is input, the process proceeds to step ST646.

For example, as illustrated in FIG. 5, the discomfort estimator learning unit 22 acquires the action pattern, for which an action information ID 171 that corresponds to the input discomfort period Δt is “a-1”, from the action/control pattern IDs 183 stored in the learning database 18. Next, as illustrated in FIG. 3, the discomfort estimator learning unit 22 refers to the action pattern having the action information ID of “a-1” from among the plurality of action patterns 173 stored in the action information database 17, and acquires a discomfort factor of “air conditioning (hot)” that is the discomfort factor 172 corresponding thereto.

Subsequently, as illustrated in FIG. 5, the discomfort estimator learning unit 22 acquires a control pattern having a control information ID of “b-2” that is stored immediately after the action pattern having the action information ID “a-1” in the action/control pattern IDs 183 stored in the learning database 18. Next, as illustrated in FIG. 4, the discomfort estimator learning unit 22 refers to the control pattern having the control information ID of “b-2” from among the plurality of control patterns 142 stored in the control information database 14, and acquires a discomfort factor of “air conditioning (hot)” that is the discomfort factor 143 corresponding thereto. In this manner, the control pattern 142 that corresponds to the discomfort factor 143 of the discomfort period Δt is input to the discomfort estimator learning unit 22.

In step ST642, the discomfort estimator learning unit 22 estimates a reaction time tx of the biological information X that indicates the heart rate variability and a reaction time ty of the biological information Y that indicates the brain wave.

In step ST643, the discomfort estimator learning unit 22 determines whether or not the reaction times tx and ty of all the pieces of biological information X and Y are estimated. If the discomfort estimator learning unit 22 determines that all the reaction times have been estimated, the process proceeds to step ST644. On the other hand, if the discomfort estimator learning unit 22 determines that not all the reaction times are estimated, the process proceeds to step ST646.

For example, as illustrated in FIG. 7, the discomfort estimator learning unit 22 checks reaction time 233 of each piece of biological information having the same user ID 231 in the estimation parameter storing unit 23, and determines that reaction time of all the pieces of biological information is estimated if the values of all the values of the reaction time 233 are other than “−1”.

In step ST644, the discomfort estimator learning unit 22 performs learning of the discomfort estimator 21 by referring to fluctuations in the biological information X and Y from the start time t1 to the end time t2 of the discomfort period Δt.

For example, as illustrated in FIG. 19, the discomfort estimator learning unit 22 sets the measurement value of the heart rate variability at the time point when the reaction time tx has elapsed from the start time t1 of the discomfort period Δt as the discomfort state threshold value Xb. Further, the discomfort estimator learning unit 22 stores the discomfort state threshold value Xb in a discomfort state threshold value 235 in association with a user ID 231 and a type of biological information 232 of the estimation parameter storing unit 23.

In addition, as illustrated in FIG. 19, the discomfort estimator learning unit 22 sets the measurement value of the brain wave at the time point when the reaction time ty has elapsed from the start time t1 of the discomfort period Δt as a discomfort state threshold value Yb. Further, the discomfort estimator learning unit 22 stores the discomfort state threshold value Yb in a discomfort state threshold value 235 in association with a user ID 231 and a type of biological information 232 of the estimation parameter storing unit 23.

In step ST645, the discomfort estimator learning unit 22 outputs a signal indicating that the learning of the discomfort estimator 21 is completed. Then, the process of the state of discomfort determination device 10 returns to step ST1.

On the other hand, in step ST646, the discomfort estimator learning unit 22 outputs a signal indicating that the learning of the discomfort estimator 21 is not completed. Then, the process of the state of discomfort determination device 10 returns to step ST1.

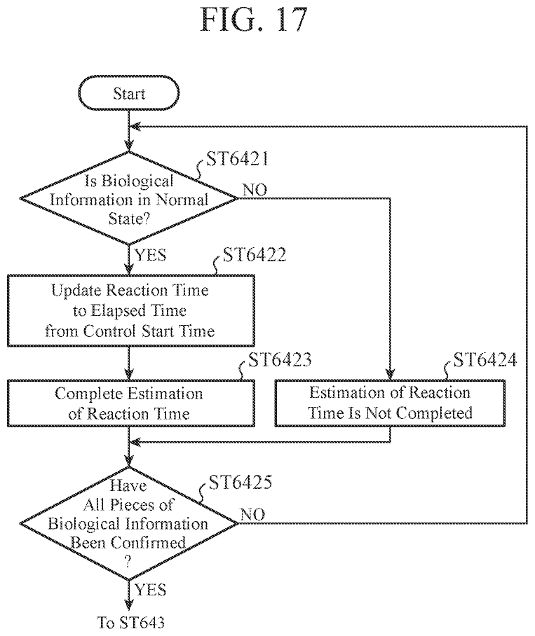

Next, the process of step ST642 will be described in more detail with reference to FIG. 17. FIG. 17 is a flowchart illustrating the operation of estimating the reaction times tx and ty in the discomfort estimator learning unit 22.

In step ST6421, the discomfort estimator learning unit 22 refers to the action/control pattern IDs 183 stored in the learning database 18 and confirms that the control pattern 142 that corresponds to the discomfort factor 143 of the discomfort period Δt is input.

Next, the discomfort estimator learning unit 22 determines whether or not the biological information X and Y is in a normal state by referring to the types of biological information 185 and the measurement values of biological information 187 stored in the learning database 18. If the discomfort estimator learning unit 22 determines that the biological information X and Y is in the normal state, the process proceeds to step ST6422. On the other hand, if the discomfort estimator learning unit 22 determines that the biological information X and Y is not normal, the process proceeds to step ST6424.

For example, as illustrated in FIG. 19, when a control pattern having a control information ID 141 of “b-2” is input, the discomfort estimator learning unit 22 determines that the biological information X is not in the normal state since the measurement value of the biological information X does not exceed the normal state threshold value Xa at a time point just after the elapse of the reaction time ty from control start time t3 when the control pattern has been input.

In step ST6424, the discomfort estimator learning unit 22 stores information indicating that estimation of the reaction times tx and ty is not completed in reaction time 233 of the estimation parameter storing unit 23. For example, as illustrated in FIG. 7, the discomfort estimator learning unit 22 stores “−1” in reaction time 233.

In step ST6425, the discomfort estimator learning unit 22 determines whether or not it is confirmed that all the pieces of biological information X and Y are in the normal state. If the discomfort estimator learning unit 22 determines that confirmation has been made for all the pieces of biological information X and Y, the processing ends. That is, the process of the discomfort estimator learning unit 22 proceeds to step ST643. On the other hand, if the discomfort estimator learning unit 22 determines that confirmation has not been made for all the pieces of biological information X and Y, the process returns to step ST6421.

For example, as illustrated in FIG. 19, in step ST6421 returned from step ST6425, the discomfort estimator learning unit 22 determines that the biological information Y is in the normal state since the measurement value of the biological information Y passes across the normal state threshold value Ya at a time point immediately after the elapse of the reaction time ty from the control start time t3.

In step ST6422, the discomfort estimator learning unit 22 updates the new reaction time ty to the elapsed time from the control start time t3 to the reaction time ty.

In step ST6423, the discomfort estimator learning unit 22 stores the updated reaction time ty as reaction time 233 in association with a type of biological information 232 in the estimation parameter storing unit 23. That is, in the discomfort estimator learning unit 22, estimation of the reaction time ty is completed.

In step ST6425, the discomfort estimator learning unit 22 determines that confirmation has been made for all the pieces of biological information X and Y, and the process ends. Then, the process of the discomfort estimator learning unit 22 proceeds to step ST643.

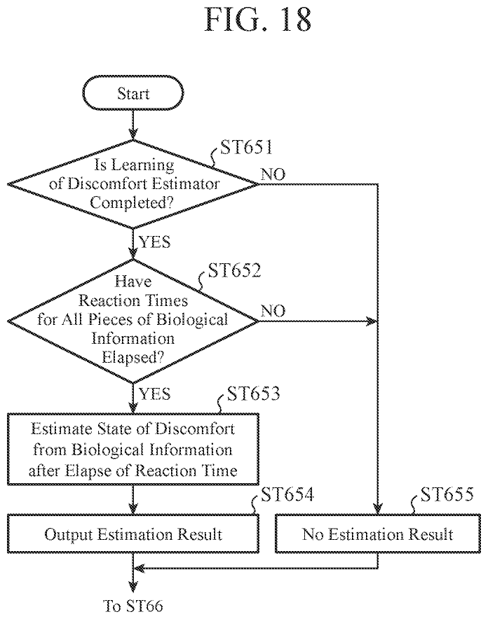

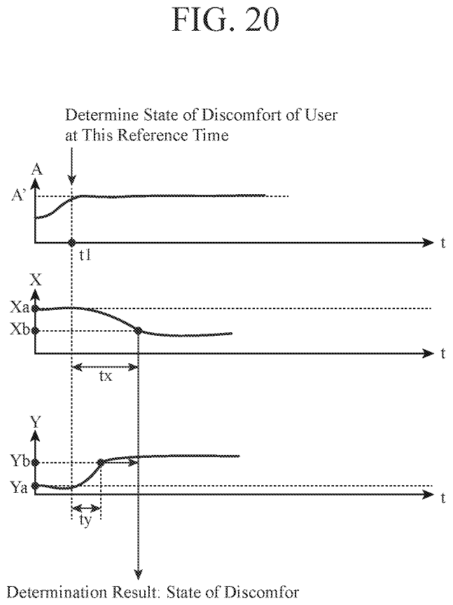

Next, the process of step ST65 will be described in more detail with reference to FIG. 18 and FIG. 20. FIG. 18 is a flowchart illustrating the operation of the discomfort estimator 21. FIG. 20 is a time chart illustrating an example of discomfort determination by the discomfort determination unit 19. Note that in FIG. 20 “t” represents time, and “T” represents temperature.

In step ST651, the discomfort estimator 21 determines whether or not learning of the discomfort estimator 21 is completed on the basis of the signal input from the discomfort estimator learning unit 22. If the discomfort estimator 21 determines that the learning is completed, the process proceeds to step ST652. On the other hand, if the discomfort estimator 21 determines that the learning is not completed, the process proceeds to step ST655.

In step ST652, the discomfort estimator 21 determines whether or not the reaction times tx and ty of all the pieces of biological information X and Y has elapsed by referring to the acquisition start time 186 stored in the learning database 18 and the reaction time 233 stored in the estimation parameter storing unit 23. If the discomfort estimator 21 determines that all the reaction times tx and ty have elapsed, the process proceeds to step ST653. On the other hand, if the discomfort estimator 21 determines that not all the reaction times tx and ty have elapsed, the process proceeds to step ST655.

Specifically, the discomfort estimator 21 extracts the longest reaction time from the reaction time 233 having the same user ID 231 in the estimation parameter storing unit 23, and determines that all the reaction times tx and ty have elapsed if the extracted reaction time 233 is longer than acquisition time required for acquisition of the biological information X and Y. Contrarily, if the extracted reaction time 233 is shorter than the acquisition time required for acquisition of the biological information X and Y, the discomfort estimator 21 determines that not all the reaction times tx and ty have elapsed.

In step ST653, the discomfort estimator 21 estimates the state of discomfort of the user on the basis of the biological information X and Y for which the reaction times tx and ty have elapsed.

For example as illustrated in FIG. 20, of the heart rate variability of the biological information X having the longest reaction time tx among the multiple pieces of biological information X and Y having the same user ID 231, the discomfort estimator 21 sets reaction start timing of the reaction time tx to the start time t1. Next, the discomfort estimator 21 acquires the latest biological information X from the learning database 18 while acquiring measurement values of the biological information Y at the time point when the reaction time ty has elapsed from the start time t1 from the learning database 18 with the lapse of time from the start time t1. Then, the discomfort estimator 21 compares the acquired measurement values of the biological information X and Y with the discomfort state threshold values Xb and Yb stored in the estimation parameter storing unit 23, respectively. At this point, the discomfort estimator 21 determines that the user is in a state of discomfort if the measurement values of the biological information X and Y exceed the corresponding discomfort state threshold values Xb and Yb.

In step ST654, the discomfort estimator 21 outputs the estimation result indicating that the user is in a state of discomfort to the discomfort determination unit 19, and the process ends. That is, the process of the state of discomfort determination device 10 proceeds to step ST66.

On the other hand, in step ST655, the process ends without the discomfort estimator 21 outputting any estimation result to the discomfort determination unit 19. That is, the process of the state of discomfort determination device 10 proceeds to step ST66.

As described above, the state of discomfort determination device 10 according to the first embodiment estimates a discomfort period Δt during which a user feels discomfort on the basis of a discomfort factor when the user's action pattern defined in advance for each type of discomfort factor matches a user's action pattern that is actually detected. Next, the state of discomfort determination device 10 estimates, as the reaction times tx and ty, time required for measurement values of the biological information X and Y to exceed normal state threshold values Xa and Ya, that is, time required for the user to transit from the state of discomfort to the normal state when the discomfort factor matches the discomfort factor that corresponds to the control information for controlling the external device. Then, the state of discomfort determination device 10 synchronizes the input timing of the biological information X and Y to the discomfort estimator 21 on the basis of the user's discomfort period Δt and the reaction times tx and ty of the biological information X and Y to estimate the user's state of discomfort.

Therefore, the state of discomfort determination device 10 can improve the accuracy of determining the user's state of discomfort by estimating individual differences in delay time of the reaction in the biological information X and Y with respect to a discomfort factor and the response strength while eliminating individual differences in the reaction speed to the discomfort factor in the biological sensors.

In addition, since the state of discomfort determination device 10 stores the user's action patterns for discomfort factors in the action information database 17 in advance, it is possible to remove the discomfort factor for the user before the user takes an action for the discomfort factor. As a result, the state of discomfort determination device 10 can improve the convenience for users.

Incidentally, in the state of discomfort determination device 10 of the above-described first embodiment, the environmental information input interface 34 includes the temperature sensor and the microphone, and the environmental information acquiring unit 11 can acquire detection results thereof; however, a humidity sensor and an illuminance sensor may be added to the environmental information input interface 34 so that the environmental information acquiring unit 11 can acquire detection results thereof as well. As a result, the state of discomfort determination device 10 can also handle the humidity and the illuminance that a user feels discomfort with.

Moreover, in the state of discomfort determination device 10, the biological information input interface 37 includes the heart rate monitor and the electroencephalograph, and the biological information acquiring unit 15 can acquire the heart rate variability and the brain wave; however, an electromyograph may be added to the biological information input interface 37 so that the biological information acquiring unit 15 can acquire an electromyogram thereof. As a result, it is possible to increase the number of types of biological information in the state of discomfort determination device 10, and thus the accuracy of determining a user's state of discomfort can be further improved.

Furthermore, in the state of discomfort determination device 10, the discomfort estimator learning unit 22 updates the discomfort state threshold value Yb on the basis of the history information of the learning database 18, and the discomfort estimator 21 determines the user's state of discomfort by comparing the discomfort state threshold values Xb and Yb and measurement values of the biological information X and Y, respectively.

At this point, if the accumulated amount of history information in the learning database 18 is sufficient, the discomfort estimator learning unit 22 performs learning of the discomfort estimator 21 by means such as machine learning using the history information, and stores parameters of the discomfort estimator 21 generated by the learning in the estimation parameter storing unit 23. Meanwhile, the discomfort estimator 21 may output an estimation result using the parameters generated by the machine learning. As a result, the state of discomfort determination device 10 can improve the accuracy of determining a user's state of discomfort even in a case where a large amount of history information is accumulated. As a method of machine learning, an approach of deep learning can be adopted, for example.

Furthermore, in the state of discomfort determination device 10, the discomfort estimator 21 sets reference time for discomfort determination on the basis of the longest reaction time tx of the biological information X; however, the discomfort estimator 21 may determine a user's state of discomfort using only the biological information Y having the shortest reaction time ty.

For example in the state of discomfort determination device 10, when the discomfort state threshold value Yb is updated on the basis of the history information in the learning database 18, the discomfort estimator learning unit 22 may update the discomfort state threshold value Yb of the brain wave only when the amount of change from the normal state threshold value Xa of the heart rate variability during the discomfort period Δt is sufficiently large, and the discomfort estimator 21 may determine the user's state of discomfort using only measurement values of the brain wave. As a result, the state of discomfort determination device 10 can shorten time elapses from when the user feels discomfort to when control to remove the discomfort factor is performed, and thus the convenience for the user can be improved.

Further, in the state of discomfort determination device 10, the discomfort estimator learning unit 22 performs only learning of the biological information X and Y, and the discomfort estimator 21 determines the state of discomfort of a user using only the biological information X and Y; however, the user's state of discomfort may be determined using the behavior information acquired by the behavior information acquiring unit 12.

For example, in the state of discomfort determination device 10, the discomfort estimator learning unit 22 may learn a threshold value indicating the degree of the behavior information acquired by the behavior information acquiring unit 12, and the discomfort estimator 21 may determine the user's state of discomfort using the threshold value. Thereby, the state of discomfort determination device 10 can detect a state of discomfort of the user from the behavior information that the user unconsciously indicates.

Note that the present invention may include a flexible combination of embodiments, a modification of any component of embodiments, or an omission of any component in embodiments within the scope of the present invention.

### INDUSTRIAL APPLICABILITY

A state of discomfort determination device according to the present invention synchronizes input timing of multiple pieces of biological information to a discomfort estimator on the basis of an estimated discomfort period of the user and reaction time of the multiple pieces of biological information, and thus it is possible to improve the accuracy of determining the user's state of discomfort, and therefore, the state of discomfort determination device is suitable for determining a state of discomfort of the user on the basis of biological information of the user.

### REFERENCE SIGNS LIST

10: state of discomfort determination device, 11: environmental information acquiring unit, 12: behavior information acquiring unit, 13: control information acquiring unit, 14: control information database, 15: biological information acquiring unit, 16: action detection unit, 17: action information database, 18: learning database, 19: discomfort determination unit, 20: discomfort period estimating unit, 21: discomfort estimator, 22: discomfort estimator learning unit, 23: estimation parameter storing unit, t: time point, Δt: discomfort period, t1: start time, t2: end time, t3: control start time, A: environmental temperature, A′: preset temperature upper limit value, X, Y: biological information, Xa, Ya: normal state threshold value, Xb, Yb: discomfort state threshold value, tx, ty: reaction time

1. A state of discomfort determination device comprising=processing circuitry
detecting action information regarding a user's action preset for each type of discomfort factor from behavior information regarding behavior that corresponds to a discomfort factor of the user;
acquiring an estimation condition for a discomfort period of the user that corresponds to the action information and estimating the discomfort period using history information that corresponds to the estimation condition;
estimating a state of discomfort of the user on a basis of multiple pieces of biological information of the user;
estimating reaction time to a discomfort factor in each of the multiple pieces of biological information on a basis of the discomfort period, and synchronizing input timing of the multiple pieces of biological information to a discomfort estimator on a basis of the discomfort period and the reaction time; and
determining the state of discomfort of the user on a basis of an estimation result of the discomfort estimator in a case where the action information is detected.

2. The state of discomfort determination device according to claim 1,
wherein the discomfort estimator estimates the state of discomfort of the user using only biological information whose reaction time is the shortest among a plurality of estimation results of the reaction time.

3. The state of discomfort determination device according to claim 1,
wherein the processing circuitry performs learning using the history information on the discomfort estimator depending on an accumulation amount of the history information.
